(19) United States
(12) Patent Application Publication
(10) Pub. No.: US 2023/0364130 A1
(43) Pub. Date: Nov. 16, 2023

(54) INHALED ARGON FOR TREATING A PATIENT SUFFERING FROM CARDIOVASCULAR DISEASE

(71) Applicants: L'Air Liquide, Sociëtë Anonyme Pour l'Etude et l'Exploitation des Procédés Georges Claude, Paris (FR); Institut National de la Sante et de La Recherche Medicale, Paris (FR); Assistance Publique - Hopitaux de Paris, Paris (FR); Université Paris est Créteil Val de Marne, Créteil (FR); Ecole Nationale Veterinaire d'Alfort, Maisons-Alfort (FR)

(72) Inventors: Géraldine FARJOT, Orsay (FR); Catherine BILLOET, Paris (FR); Matthieu CHALOPIN, Etampes (FR); Quentin DE ROUX, Créteil (FR); Renaud TISSIER, Maisons Alfort (FR); Nicolas MONGARDON, Créteil (FR)

(21) Appl. No.: 18/028,164

(22) PCT Filed: Sep. 13, 2021
(86) PCT No.: PCT/EP2021/075099

§ 371 Date: Mar. 23, 2023

(30) Foreign Application Priority Data
Sep. 23, 2020 (FR) 2009617

(51) Int. Cl. A61K 33/00; A61K 9/00; A61P 9/10

(52) U.S. Cl. CPC A61K 33/00; A61K 9/0073; A61P 9/10

(57) ABSTRACT

The invention concerns a use of argon gas administered by inhalation in order to directly protect or preserve haemodynamics and to prevent cardiovascular failure by controlling the blood flow, blood pressure and blood diffusion throughout the body of a human patient suffering from cardiovascular disease requiring heart or vascular surgery, in particular an aneurysm of the aorta, a myocardial ischaemia, a severe heart failure or a cardiopathy requiring a surgical operation. The argon is preferably mixed with oxygen.

### CROSS REFERENCE TO RELATED APPLICATIONS

This application is a 371 of International Application No. PCT/EP2021/075099, filed Sep. 13, 2021, which claims priority to French Patent Application No. 2009617, filed Sep. 23, 2020, the entire contents of which are incorporated herein by reference.

### BACKGROUND

The invention relates to a use of argon gas administered by inhalation in order to directly protect or preserve hemodynamics and to prevent cardiovascular failure by controlling blood circulation, blood pressure and blood diffusion throughout the body of a human patient suffering from cardiovascular disease requiring heart or vascular surgery.

Several cardiovascular diseases can be the cause of hemodynamic disturbances and of cardiac decompensation, in particular:
diseases of the aorta which is responsible for bringing blood from the heart to the rest of the body. Thus, an aneurysm of the thoracic aorta or the abdominal aorta and dissection of the aortic arch can produce serious consequences for the other organs. These are medical emergencies treated by surgical techniques aimed at replacing the aorta with a medical device, such as a synthetic Dacron tube;
myocardial ischemia or infarction is a necrosis of the heart resulting from poor blood perfusion of the heart. The treatment thereof aims to restore the perfusion to the arteries;
certain types of heart failure require surgical treatments of different types, in particular replacement of a heart valve, correction of coronary vessels blocked by atherosclerotic lesions, fitting of a pacemaker, or even sometimes extracorporeal blood circulation;
cardiopulmonary transplants.

During the intra-operative and/or post-operative period of these cardiovascular diseases, cardiogenic shock characterized by a fall in heart blood flow is often observed, as are other hemodynamic deteriorations, resulting in a loss of blood perfusion efficiency and possible subsequent irreversible damage to one or more organs, such as kidneys, liver, intestines, pancreas, lungs, heart, limbs, brain, etc., associated with a high risk of mortality.

In view of this, the problem is therefore that of being able to maintain, that is to say preserve and/or protect, the hemodynamics, in particular the heart blood flow, that is to say the cardiac function, the cardiovascular hemodynamics and efficient blood perfusion of the abdominal organs, in a patient (or patients) suffering from cardiovascular disease requiring heart or vascular surgery so as to avoid or minimize all or part of the aforementioned problems and to prevent the harmful effects of cardiogenic shock.

### SUMMARY

A solution according to the invention relates to an inhalable gaseous medicament containing argon gas for use in directly preserving and/or protecting hemodynamics and in preventing cardiovascular failure by controlling blood circulation, blood pressure and diffusion of blood throughout the body, in particular the heart blood flow, of a human patient, i.e. an individual, suffering from cardiovascular disease requiring heart or vascular surgery, wherein the inhalable gaseous medicament is intended to be administered to said patient by inhalation before the start of the heart or vascular surgery.

The argon-based gaseous medicament of the invention is intended to act directly on the hemodynamic parameters, in addition to and independently of any anti-ischemic action, so as to obtain direct prevention of the cardiovascular failure that may occur in the context of the cardiovascular disease in question.

Thus, the inhaled argon, by virtue of its action on the vascular system and maintenance of cardiac output, makes it possible to ensure good perfusion pressure, thus reducing the need for recourse to additional treatments, to prevent any cardiovascular failure.

In the context of the invention:
“preservation of hemodynamics” is intended to mean preservation of the physiological functions, i.e. cardiovascular functions, and the control thereof by the autonomic nervous system, which allow control of blood circulation, blood pressure and blood diffusion throughout the body, in particular heart blood flow, heart rate, right and left ventricular ejection pressures, vascular resistance and organ blood perfusion;
“directly preserving and/or protecting hemodynamics” is intended to mean a direct effect resulting in particular in a reduction in pulmonary vascular resistance and an increase in right cardiac output but also in left cardiac output; with improvement of the aforementioned hemodynamic status, this being in addition to and/or independently of any anti-ischemic action.

In other words, the invention relates to an inhalable gaseous medicament containing argon gas for use in a method for directly preserving and/or protecting hemodynamics and preventing cardiovascular failure by controlling blood circulation, blood pressure and blood diffusion throughout the body, in particular for protecting heart blood flow, of a human patient suffering from cardiovascular disease requiring heart or vascular surgery; said method comprising:
administration of said gaseous medicament to said patient by inhalation,
followed by heart or vascular surgery,
and wherein the administration of said gaseous medicament:
begins before the heart or vascular surgery begins, and
continues throughout the heart or vascular surgery.

Depending on the embodiment under consideration, the gaseous medicament of the invention may comprise one or more of the following features:
the cardiovascular disease is an aortic aneurysm, myocardial ischemia, severe heart failure or cardiopathy requiring heart or vascular surgery;
it contains argon and oxygen, in particular an Ar/O₂ binary mixture;
the oxygen content is at least 21% by volume (vol %), preferably at least 25 vol %;
the oxygen content is at least 30 vol %;
the argon content is at least 30 vol %, preferably at least 40 vol
the argon content is between 50 and 70 vol %;
it also contains nitrogen or air;
the administration of said gaseous medicament continues after heart or vascular surgery;
the heart or vascular surgery comprises anesthesia of the patient, in particular general anesthesia;
the general anesthesia of the patient is liable to cause, in the absence of administration of argon, a reduction in the cardiac output, in particular of at least 10%, or even more;
the heart or vascular surgery comprises establishing extracorporeal circulation or ECC, or extracorporeal blood oxygenation, in particular extracorporeal membrane oxygenation, or ECMO;
the heart or vascular surgery comprises establishing ECC with clamping of the aorta, in particular at the supraceliac or suprarenal level;
the vascular surgery comprises repair of an aneurysm of a large blood vessel, in particular the aorta;
the vascular surgery comprises repair of an aneurysm of a large blood vessel with clamping of said large blood vessel liable to cause hemodynamic disturbances, in particular an increase in cardiac afterload with a drop in cardiac output;
the protection of heart blood flow comprises maintenance of cardiac output with an at least 20% attenuated decrease immediately after the start of heart or vascular surgery, in particular as soon as any clamping takes place, compared to surgery performed under the same conditions but without administration of argon gas;
the protection of heart blood flow comprises maintaining heart blood flow by improving the conditions of left ventricular pressure, in particular left ventricular diastolic pressure; or left ventricular preload, in particular related to an effect on pulmonary circulation, and/or possibly right ventricular pressure;
the protection of heart blood flow comprises maintaining heart blood flow by acting on the pulmonary and/or systemic circulation;
the protection of heart blood flow comprises maintaining the hemodynamics, in particular the heart blood flow, through the dilating action of argon on all or part of the blood vessels, in particular the pulmonary blood vessels;
the argon makes it possible to prevent all or some of the harmful effects of at least one anesthetic agent used for general anesthesia of the patient;
general anesthesia comprises administering to the patient at least one anesthetic substance, for example halothane, isoflurane, desflurane, sevoflurane, thiopental, propofol, fentanyl, midazolam, remifentanil, sufentanil or nitrous oxide (i.e. N₂O);
the gaseous medicament contains argon as active ingredient;
the patient is a human being, namely a man or a woman, whether a child, an adolescent or an adult, including the elderly;
the gaseous medicament containing argon is packaged in a pressurized gas container, such as a pressurized gas cylinder;
alternatively, the gaseous medicament is packaged in the form of pure argon and the gas mixture is obtained or produced by extemporaneous mixing of the compounds (e.g. Ar and O₂) at the site of use by means of a gas mixer, an anesthesia ventilator or any other suitable gas delivery device;
the mixture or the pure gas is obtained extemporaneously and then administered to the patient by means of an anesthesia ventilator configured to allow administration of this gas mixture;
the gaseous medicament is suitable for administration by inhalation at an administration flow rate of between 1 and 30 l/min. The invention also relates to the use of an Ar/O₂ gas mixture₂ containing argon (Ar) and oxygen (O₂) (mol %) according to the invention, for producing an inhalable medicament which makes it possible to directly preserve and/or protect hemodynamics and to prevent cardiovascular failure by controlling blood circulation, blood pressure and blood diffusion throughout the body, in particular to protect heart blood flow, of a human patient suffering from cardiovascular disease requiring heart or vascular surgery, said inhalable medicament being formulated for being and intended to be administered to the patient by inhalation, before the start of heart or vascular surgery, and then throughout the heart or vascular surgery, or even also after the heart or vascular surgery has ended.

Furthermore, the invention also relates to a treatment method which makes it possible to directly preserve and/or protect hemodynamics and to prevent cardiovascular failure by controlling blood circulation, blood pressure and blood diffusion throughout the body, in particular to protect heart blood flow, of a human patient suffering from cardiovascular disease requiring heart or vascular surgery, wherein:
a) a patient who must undergo heart or vascular surgery is selected,
b) an Ar/O₂ gas mixture according to the invention is administered to said patient by inhalation before and during, or even after, heart or vascular surgery.

Depending on the embodiment under consideration, the treatment method of the invention can comprise one or more of the following features:
the gas mixture is administered at a flow rate of between 1 and 30 l/min;
the patient is an adult; an adolescent or a child;
the gas mixture is administered via a breathing interface for supplying gas to the patient, for example an intubation system or a breathing mask;
the gas mixture is packaged in a gas container fitted with a gas distribution valve unit, preferably an integrated pressure regulator unit or IPR;
the gas container has a cylindrical body;
the gas container has a cylindrical body made of metal or of composite materials, in particular made of aluminum alloy or of steel;
the gas container is fitted with a gas distribution valve unit protected by a protective cover, also called a “cap”, made of polymer or of metal;
the breathing interface is fluidically connected to the gas container by a flexible hose or the like for conveying the gas;
the breathing interface is fluidically connected to a medical ventilator supplied by the gas container, via one or more flexible hoses or the like for conveying the gas.

### BRIEF DESCRIPTION OF THE DRAWINGS

The invention will now be better understood from the following detailed description given as an illustrative but nonlimiting example and with reference to the appended figures, wherein:

FIG. 1 shows diagrammatically the cardiac output measured by a cardiac output valve before, during and after aortic ischemia;

FIG. 2 shows diagrammatically the norepinephrine dose required to maintain a stable arterial pressure following reperfused aortic ischemia;

FIG. 3 shows diagrammatically the histology score of several organs; and

FIG. 4 shows diagrammatically the variations in pulmonary vascular resistance according to the different levels of suprarenal then supraceliac clamping, during surgery on rabbits.

### DETAILED DESCRIPTION OF THE PREFERRED EMBODIMENTS

### Experimental Protocol

The efficacy of an administration of argon gas according to the invention has been demonstrated in an experimental model in which interruption of abdominal circulation is provoked in rabbits, namely male rabbits of the New Zealand breed.

More specifically, the model used is a lagomorphic model of multiple organ failure by clamping the supraceliac aorta.

The rabbits were sedated by intravenous injection (Zoletil® 15 mg/kg), then intubated to be ventilated. Anesthesia is completed by intravenous administration of thiopental (10 mg/kg) and methadone hydrochloride (Comfortan® 0.6 mg/kg); and maintained as needed with thiopental boluses.

After a period of hemodynamic stabilization, the supraceliac aorta is clamped with an atraumatic clamp for 30 minutes. In order to avoid any thrombus formation at the level of the clamping, a bolus of unfractionated heparin (250 IU/kg) is administered to the rabbit in question, 5 minutes before clamping.

The effectiveness of the clamping is verified by the absence of an aortic pulse downstream of the clamping. Similarly, the absence of thrombus is assessed by the presence of an aortic pulse after unclamping.

The rabbits are separated into 2 groups:
a Control Group (CG) inhaling an N₂/O₂ binary gas mixture containing 30% of oxygen and the rest as nitrogen (vol %);
an Argon Group (AG) according to the invention inhaling an Ar/O₂ binary gas mixture containing 30% of oxygen and the rest as argon (vol %), i.e. 70 vol % of argon.

### Cardiac Output Protection

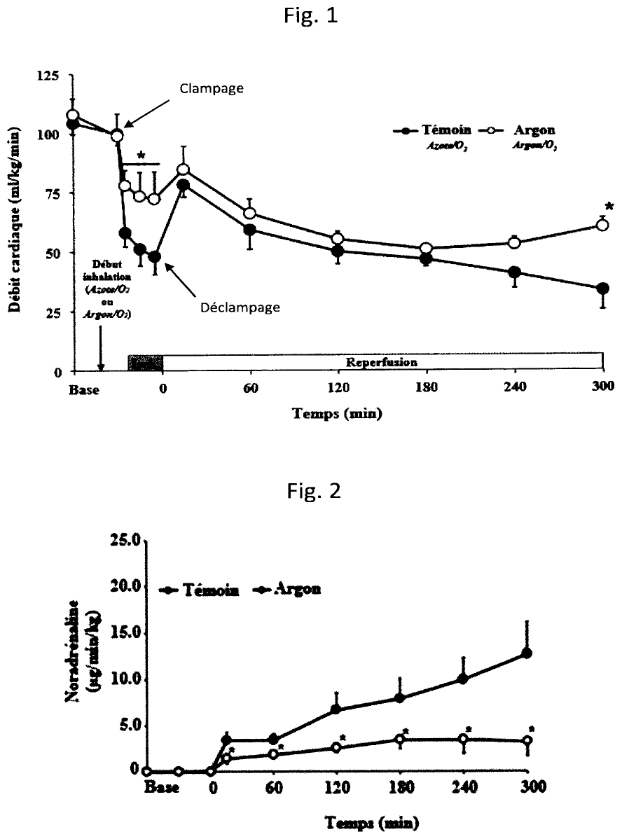

The results obtained are illustrated in FIG. 1.

The cardiac output is measured by a cardiac output valve before, during and after ischemia (Isch.) of the aorta.

The moments of clamping and unclamping of the aorta which correspond to cardiovascular surgery are indicated in FIG. 1.

As is seen, at the start of the experiment, no significant difference in cardiac output is observed between the Control and Argon Groups: 104±5 ml/kg/min for CG versus 108±7 ml/kg/min for AG.

Similarly, after 30 minutes of gas inhalation (i.e. before clamping), no significant difference in cardiac output is observed between the Control and Argon Groups: 99±5 ml/kg/min for CG versus 99±10 ml/kg/min for AG.

On the other hand, during the damping phase (which mimics the cardiovascular surgery phase), the output is significantly less reduced in the Argon Group than in the Control Group. More specifically, maintenance of cardiac output is observed in FIG. 1 with an approximately 20% attenuated decrease immediately after the beginning of surgery, in particular upon damping, resulting in protection of heart blood flow in the presence of argon.

As explained below in the Additional Experiments, this maintenance of heart blood flow results in particular from an improvement in left ventricular pressure conditions in the presence of argon and from an effect of the argon on pulmonary circulation.

Next, after unclamping, during the reperfusion phase (i.e. phase following surgery), a slow and continuous decrease in cardiac output was observed in both groups, with no significant difference between the groups up to 240 minutes of reperfusion. However, after 240 minutes, cardiac output continued to decrease in the Control Group while it stabilized in the Argon Group. The cardiac output was thus significantly different between the two groups after 300 minutes of reperfusion: 32±9 ml/kg/min for CG versus 60±4 ml/kg/min for AG.

Through its action on cardiac output, argon allows, via the arterial network, a better blood distribution and therefore a better organ perfusion.

Thus, surprisingly, since argon has no effect on the basal hemodynamic parameters, it makes it possible, under conditions of cardiogenic-type stress, to maintain cardiac output and therefore good organ perfusion. This has resulted in a better general condition of the animals and maintenance of normal acid-base biochemical parameters in the argon-treated group. In particular, the pH, HCO₃⁻ and lactate parameters are significantly preserved within normal values in the Argon Group compared to the Control Group. Thus, after 300 minutes of reperfusion, the pH is maintained at 7.16 in the Argon Group, against a drop to 6.91 in the Control Group.

### Other Hemodynamic Parameters

Heart rate remained stable throughout the protocol, with no significant difference between the groups.

During the ischemia (inch) phase following clamping, a sudden increase in mean arterial pressure was observed but with no significant difference between the groups: 124±7 mmHg for CG and 130±7 mmHg for AG.

During the reperfusion phase, i.e. after unclamping, the mean arterial pressures remained stable with no significant difference between the groups. However, to maintain these mean arterial pressure values, norepinephrine doses were significantly lower in the Argon Group (AG) than in the Control Group (CG) throughout reperfusion.

The norepinephrine doses required to maintain a stable arterial pressure following reperfused aortic ischemia (at T0) are shown in FIG. 2.

It is seen therein that, after 15 minutes of reperfusion, the norepinephrine doses are 3.4±1.0 μg/kg/min in the Control Group and 1.4±0.6 μg/kg/min in the Argon Group. At the end of the 300-minute monitoring, the norepinephrine requirements reach 12.6±3.0 μg/kg/min in the Control Group compared to 3.3±1.6 μg/kg/min in the Argon Group.

At the end of the reperfusion phase, the inhaled argon, by virtue of its action on the vascular system and maintenance of cardiac output, makes it possible to maintain good perfusion pressure, thus reducing the need for recourse to additional treatments, to prevent any cardiovascular failure.

### Histological Analyses

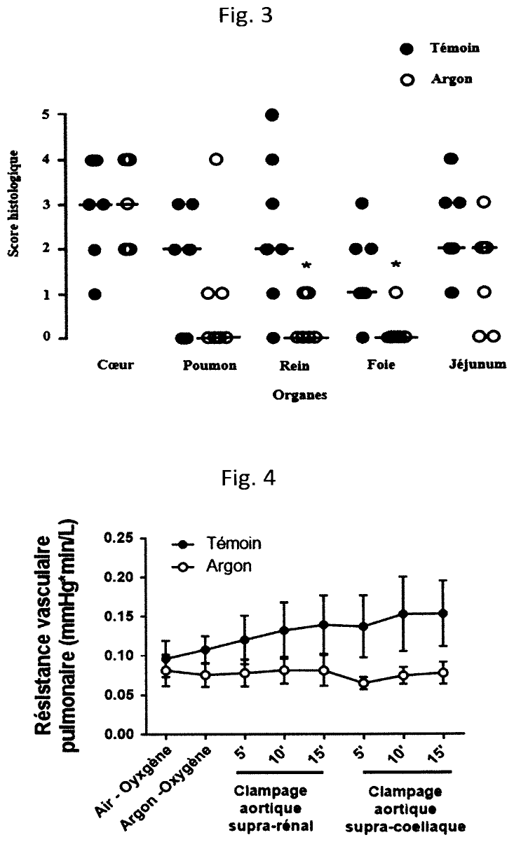

FIG. 3 represents the histology score of several organs: heart, liver, lungs, kidneys and jejunum (i.e. intestines).

In the myocardium, similar subendocardial necrosis lesions were observed in the two groups, with no significant difference. Although congestive and edematous lesions of the lung parenchyma were more severe in the Control Group than in the Argon Group, the difference was not significant.

Conversely, the tubular necrosis lesions are significantly greater in the Control Group than in the Argon Group.

Concerning centrilobular clarification in the liver, the treated rabbits have a histology score significantly lower than in the Control Group. Finally, no difference was observed in jejunal lesions.

Argon demonstrates, under conditions of interrupted circulation, a protective effect on the noble organs, essential to life, downstream of the interrupted arterial network. In other words, argon, by maintaining the cardiac output and therefore the perfusion of the organs, consequently makes it possible to maintain in good condition the noble organs essential to life downstream of the interrupted arterial network.

### Additional Experiments Showing the Direct Hemodynamic Action of Argon

In order to demonstrate the existence of a direct hemodynamic effect of argon on the state of shock, obtained in addition to its possible indirect effects, in particular its possible anti-ischemic and/or possible organ-protecting effects, an additional preliminary study was carried out on 4 control rabbits and 4 rabbits exposed to argon.

A first clamping of the aorta of these rabbits was performed above the renal artery, followed by clamping at the supraceliac level. The objective is to see the effect of argon during a gradual modification of systemic resistance.

FIG. 4 shows the calculated pulmonary vascular resistances in 4 animals exposed to oxygen-enriched air compared to 4 animals exposed to a mixture of argon (70 vol %) and oxygen (30 vol %) during ascending aortic clamping.

It is seen therein that the effect on cardiac output is attenuated by this gradual nature of the hemodynamic modifications, but also that pulmonary vascular resistance is indeed immediately reduced in animals exposed to argon, thus demonstrating that argon acts by improving the hemodynamic status via a direct effect.

### Conclusion

In the experimental model of aortic clamping in rabbits having been implemented within the framework of the invention, inhalation of argon allows maintenance of the hemodynamic parameters, direct prevention of cardiovascular failure by direct attenuation of the decrease in cardiac output, of the state of shock, this being in addition to and independently of anti-ischemic effects on renal and hepatic lesions.

Inhalation of argon makes it possible to limit the harmful multiple organ consequences linked to cardiogenic shock in this model of abdominal aortic clamping, mimicking the picture presented by patients after abdominal vascular surgery. This benefit follows biphasic kinetics with improved cardiac output during the aortic clamping phase, then a late beneficial effect at the end of the experimental monitoring.

After unclamping, the cardiac output is in fact initially similar in the two groups then significantly higher after 300 minutes of reperfusion in the group treated with argon according to the invention.

An attenuation of the state of vasoplegic shock secondary to the ischemia-reperfusion phenomenon, with a significant drop in the norepinephrine doses and a less disturbed acid-base balance in the blood, was also demonstrated in the group treated with argon according to the invention.

These trials show the efficacy of argon inhalation for maintaining the hemodynamic parameters, cardiac output and good organ perfusion under conditions mimicking the interruption of circulation occurring during a surgical procedure linked to heart or vascular disease, this being independently of any ischemic effect possibly present.

In other words, inhalation of argon makes it possible to reduce the risk of cardiogenic shock liable to have fatal consequences for the patient during heart or vascular surgery procedures.

According to the invention, a gaseous medicament based on argon gas, administered by inhalation, is therefore used at least before and during cardiovascular surgery, to directly protect or preserve the hemodynamics, in particular heart blood flow, and to prevent any cardiovascular failure by controlling blood circulation, blood pressure and diffusion of blood throughout the body of a human patient suffering from a cardiovascular disease requiring heart or vascular surgery.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed.

Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something, The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A method of stabilizing hemodynamics and reducing the risk of cardiovascular failure by controlling blood circulation, blood pressure and diffusion of blood throughout the body of a human patient suffering from cardiovascular disease requiring a heart or vascular surgery, the method comprising a step of administering an inhalable gaseous medicament containing argon gas directly to said patient by inhalation before a start of the heart or vascular surgery.

2. The method as claimed in claim 1, characterized in that the cardiovascular disease is an aortic aneurysm, myocardial ischemia, severe heart failure or a cardiopathy requiring surgical intervention.

3. The method as claimed in claim 1, characterized in that the inhalable gaseous medicament contains argon and oxygen.

4. The method as claimed in claim 3, characterized in that the oxygen content is at least 21% by volume (vol %).

5. The method as claimed in claim 3, characterized in that the oxygen content is at least 30 vol %.

6. The method as claimed in claim 3, characterized in that the argon content is at least 30 vol %.

7. The method as claimed in claim 3, characterized in that the inhalable gaseous medicament also contains nitrogen or air.

8. The method as claimed in claim 1, characterized in that the heart or vascular surgery comprises extracorporeal circulation (ECC) or extracorporeal blood oxygenation via extracorporeal membrane oxygenation (ECMO).

9. The method as claimed in claim 1, characterized in that the heart or vascular surgery comprises establishing ECC with clamping of the aorta.

10. The method as claimed in claim 1, characterized in that the vascular surgery comprises repair of an aneurysm of a large blood vessel.

11. The method as claimed in claim 10, characterized in that the vascular surgery comprises repair of an aneurysm of a large blood vessel with clamping of said large blood vessel.

12. The method as claimed in claim 1, characterized in that the controlling blood circulation, blood pressure and diffusion of blood comprises maintenance of cardiac output with an at least 20% attenuated decrease immediately after the start of heart or vascular surgery.

13. The method as claimed in claim 12, characterized in that the controlling blood circulation, blood pressure and diffusion of blood comprises maintaining heart blood flow by improving the conditions of left ventricular pressure.

14. The method as claimed in claim 1, characterized in that the controlling blood circulation, blood pressure and diffusion of blood comprises maintaining heart blood flow by acting on a pulmonary or a systemic circulation.

15. The method as claimed in claim 1, characterized in that the controlling blood circulation, blood pressure and diffusion of blood comprises maintaining the hemodynamics, through a dilating action of argon on all or part of a blood vessel.
